(19) United States
(12) Patent Application Publication
(10) Pub. No.: US 2025/0169984 A1
(43) Pub. Date: May 29, 2025

(54) DEVICES AND METHODS FOR TREATING OCULAR CONDITIONS

(71) Applicant: APPLIED TISSUE TECHNOLOGIES LLC, Hingham, MA (US)

(72) Inventors: Elof Eriksson, East Plainfield, MA (US); Michael Broomhead, Scituate, MA (US)

(21) Appl. No.: 19/036,608

(22) Filed: Jan. 24, 2025

Related U.S. Application Data
(63) Continuation of application No. 17/626,041, filed on Jan. 10, 2022, now abandoned.
(60) Provisional application No. 62/873,407, filed on Jul. 12, 2019.

(51) Int. Cl. A61F 9/00; A61F 9/04

(52) U.S. Cl. CPC A61F 9/0008; A61F 9/04

(57) ABSTRACT

Ocular treatment devices are disclosed. An ocular treatment device may include a chamber defining an isolated treatment space, a sealing portion, and a port configured to enable introduction of a therapeutic agent to the isolated treatment space. An ocular treatment device may include a chamber having an upper assembly, central assembly, and lower assembly, and a sealing portion. Related methods of treating various ocular conditions with such devices are also disclosed.

### CROSS-REFERENCE TO RELATED APPLICATIONS

This application is a continuation of U.S. patent application Ser. No. 17/626,041, filed on Jan. 10, 2022, which is a U.S. national phase application and claims the benefit of priority under 35 U.S.C. § 371 to International (PCT) Patent Application Serial No. PCT/US2020/041636 filed on Jul. 10, 2020 which, in turn, claims the benefit of priority to U.S. Provisional Patent Application Ser. No. 62/873,407 filed on Jul. 12, 2019, the entire disclosure of each of which is hereby incorporated herein by reference in its entirety for all purposes.

### STATEMENT REGARDING FEDERALLY SPONSORED RESEARCH OR DEVELOPMENT

This invention was made with government support under AIBS No. 140024 awarded by the U.S. Army Medical Research and Material Command (MRMC) Clinical and Rehabilitative Medicine Research Program (PAD5). The government has certain rights in the invention.

### BACKGROUND

The number of ocular and periocular injuries is on the rise, particularly in connection with the military. Eye injuries are associated with a high burden and cost of care. Subjects may face extensive hospitalization and there is generally a significant impact on quality of life and productivity.

Infection of the cornea or infectious keratitis (IK) is an ophthalmic emergency and can progress to blindness if not promptly and adequately treated. Caused by bacterial, viral, fungal, and parasitic agents, IK is a major contributor to corneal opacification and blindness worldwide. Complications from IK develop quickly and can lead to ulceration of the cornea, neovascularization, corneal scarring and opacification, perforation, endophthalmitis, and in the most severe cases, blindness and/or eye loss. Due to the potential for these serious sequelae, infectious keratitis remains a leading cause of blindness globally, second only to cataracts. Current approaches for the treatment of various ocular conditions, such as IK, are insufficient.

### SUMMARY

In one aspect, an ocular treatment device is provided. The ocular treatment device may comprise a chamber configured to enclose a region spanning both eyes of a subject, a sealing portion at a base of the chamber configured to be secured at the periphery of the chamber around the region spanning both eyes of the subject form the isolated treatment space, and a port in the chamber configured to enable fluid communication with the isolated treatment space. The chamber may include an interior surface defining an isolated treatment space.

In another aspect, an ocular treatment device is provided. The ocular treatment device may comprise a chamber configured to enclose an eye region of a subject. The chamber may comprise an upper assembly comprising a port, a central assembly connected to a lower surface of the upper assembly, and a lower assembly connected to a lower surface of the central assembly. The lower assembly may have a longest linear dimension that is greater than that of the central assembly. The upper assembly, central assembly, and lower assembly together may form an interior surface defining an isolated treatment space. The ocular treatment device may further comprise a sealing portion at the lower assembly of the chamber configured to be secured as an orbital rim around an eye of the subject to form the isolated treatment space.

In some embodiments, the port in the chamber is configured to enable fluid communication with the isolated treatment space. In some embodiments, the central assembly is configured to contain a volume of a liquid delivered through the port.

In some embodiments, the eye region comprises an eye and eyelid of the subject. In further embodiments, the ocular treatment device may comprise a tube connected to the port and in fluid communication with the isolated treatment space. The tube may be configured to remove fluid from, or introduce fluid to, the isolated treatment space. In further embodiments, the port may be configured to enable negative pressure therapy within the isolated treatment space.

In some embodiments, the interior surface of the chamber is characterized by a plurality of embossed structures. The embossed structures may be configured to directly contact the eyelid of the subject. In some embodiments, the embossed structures may be configured to create pathways between the interior surface of the chamber and the eye region of the subject.

In some embodiments, the plurality of embossed structures may be positioned at a distance of about 0.2 mm to about 10 mm apart from one another. In some embodiments, the plurality of embossed structures may have a height of about 0.1 mm to about 5 mm. The plurality of embossed structures may be positioned in a uniform pattern on the interior surface of the chamber. In some embodiments, each of the plurality of embossed structure has a shape selected from the group consisting of a cone, a pyramid, a pentagon, a hexagon, a half sphere, a dome, a rod, an elongated ridge with round sides, and an elongated ridge with square sides. In some embodiments, the plurality of embossed structures may be semi-rigid. In some embodiments, the plurality of embossed structures may cover about 50 to about 100 percent of the interior surface of the chamber.

In some embodiments, the chamber is configured to provide an in vivo, tissue culture-like condition in the isolated treatment space. For example, the chamber may be configured to provide an isolated treatment space that is characterized by a moist environment.

In some embodiments, the chamber may be constructed and arranged to treat both the eye and periorbital tissue of the subject.

In some embodiments, the sealing portion comprises a mechanical or an adhesive seal. In some embodiments, the sealing portion may be configured to adhere to compromised tissue and/or burned tissue. In some embodiments, an adhesive of the adhesive seal is substantially breathable. For example, the adhesive of the adhesive seal may be an acrylic seal.

In some embodiments, the chamber may be constructed of a substantially conformable material. In some embodiments, the chamber may be constructed of a material characterized by a predetermined oxygen permeability level. For example, the chamber may be constructed of a material that promotes oxygen transmissibility across a cornea of the subject. In some embodiments, the chamber may be constructed of a semi-transparent or substantially transparent material. In some embodiments, the chamber may be constructed of a material that is treated. In other embodiments, the chamber may be formed from a single sheet of material. In particular embodiments, the chamber may be constructed of polyurethane.

In some embodiments, a shape of the chamber may be substantially oblong. In some embodiments, the chamber may be configured to enclose both eyes of the subject.

In certain embodiments, the ocular treatment device as described herein may be integrated into a facial wound chamber.

In some embodiments, the port is self-sealing. In some embodiments, the isolated treatment space is characterized by an optimized shape or volume. For example, the isolated treatment space may be configured to hold a predetermined volume and/or concentration of a therapeutic agent. The chamber may be configured to maintain a therapeutic agent in contact with the eye and/or periorbital tissue of the subject. In some embodiments, the isolated treatment space may be configured to immerse the eye and/or periorbital tissue of the subject in a therapeutic agent. In certain embodiments, the chamber may be configured to protect and/or prevent ocular injury.

In some embodiments, the ocular treatment deice may be configured to be removable and/or resealable. In some embodiments, the chamber is provided in substantially sterile packaging. In some embodiments, the chamber is disposable and/or configured for single use.

In some embodiments, the chamber does not comprise a porous insert.

In further embodiments, the ocular treatment deice may include at least one sensor constructed and arranged to detect and/or monitor at least one of an oxygen level, temperature, and pH level within the isolated treatment space. A temperature sensor may comprise a plurality of filamentary structures, e.g., metallic filamentary structures, disposed onto a flexible substrate. An oxygen sensor may comprise an optical oxygen sensor, e.g., a sensor including at least one light source and a photodetector, disposed on a flexible substrate. A pH sensor may comprise at least one pH-sensitive dye incorporated into a polymer membrane and a photodetector. In some embodiments, the at least one sensor may be constructed and arranged to transmit collected data wirelessly.

In further embodiments, the ocular treatment deice may include at least one of a fluid trap, a pump, an exhaust port, and a suction device.

In some embodiments,, the ocular treatment deice may be characterized by a bellows structure.

In further embodiments, the ocular treatment deice may be configured for clinical use in conjunction with a protective shield.

In accordance with another aspect, there is provided a method of treating bacterial keratitis in a subject. The method may comprise steps of securing an ocular treatment device as described herein over a periorbital region of a subject and introducing a therapeutic agent to the isolated treatment space of the ocular treatment device chamber to treat the bacterial keratitis of the subject.

In some embodiments, the bacterial keratitis comprises Pseudomonas aeruginosa keratoconjunctivitis.

In some embodiments, the therapeutic agent is provided in a vehicle formulated as a liquid, suspension, gel, hydrogel, ointment, or foam. The therapeutic agent may be formulated for sustained release. In some embodiments, the therapeutic agent is an antifungal, antibiotic, anti-inflammatory, or analgesic agent. In particular example, the antibiotic may be moxifloxacin.

In further embodiments, the method may include promoting a wet or moist environment in the isolated treatment space. For example, the method may include immersing a cornea of the eye of the subject in a fluid environment within the isolated treatment space. In some embodiments, treatment may improve corneal integrity. In some embodiments, treatment may reduce corneal desiccation and/or corneal scarring.

In accordance with another aspect, there is provided a method of treating an ocular condition in a subject. The method may comprise steps of securing an ocular treatment device as described herein over a periorbital region of a subject and introducing a therapeutic agent to the isolated treatment space of the ocular treatment device chamber to treat the ocular condition of the subject.

In accordance with another aspect, there is provided a method of treating an ocular condition in a subject. The method may comprise steps of securing an ocular treatment device as described herein over a periorbital region of a subject and introducing a therapeutic agent to the isolated treatment space of the device chamber to treat the ocular condition of the subject, the therapeutic agent delivered at a concentration of about 10 to about 1000 times MIC.

In some embodiments, the ocular condition is an ocular infection. For example, the infection may comprise bacterial or viral keratitis. In particular embodiments, the ocular infection comprises Pseudomonas aeruginosa keratitis.

In some embodiments, the ocular condition may be an ocular injury. For example, the ocular injury may pertain to any of damaged periorbital tissue, an eyelid injury, and/or a corneal epithelial wound.

In some embodiments, the ocular condition pertains to any of a corneal ulcer, exposure keratopathy, a facial burn, and/or a blepharoplasty procedure. In some embodiments, the subject may have a compromised periocular structure.

In further embodiments, the methods disclosed herein may include providing a skin graft on an eyelid of the subject to reestablish eyelid coverage. The skin graft may be a partial thickness skin graft or a full thickness skin graft.

In some embodiments, the therapeutic agent is provided in a vehicle formulated as a liquid, suspension, gel, hydrogel, ointment, or foam. The therapeutic agent may be formulated for sustained release. In some embodiments, the therapeutic agent comprises an antimicrobial agent. In some embodiments, the therapeutic agent may be an antifungal, antibiotic, anti-inflammatory, or analgesic agent. In particular embodiments, the antibiotic may be vancomycin or moxifloxacin.

In some embodiments, the therapeutic agent may be delivered at a concentration of about 10 to about 1000 times MIC. For example, the therapeutic agent may be delivered at a concentration of up to 100 times MIC. In particular embodiments, the therapeutic agent may be delivered at a concentration of at least 100 times MIC.

In further embodiments, the methods disclosed herein may include promoting a wet environment in the isolated treatment space. For example, the method may include immersing a cornea of the eye of the subject in a fluid environment within the isolated treatment space.

In further embodiments, the methods disclosed herein may include introducing oxygen to the device chamber.

In further embodiments, the methods disclosed herein may include introducing a saline solution or a media solution to the device chamber.

In further embodiments, the methods disclosed herein may include a growth factor and/or cells to the device chamber.

In some embodiments, treatment may improve corneal integrity. In some embodiments, treatment may reduce corneal desiccation and/or corneal scarring. In some embodiments, treatment may slow or prevent progression of ocular wound depth. In some embodiments, surrounding tissue and/or tissue under an adhesive rim of the ocular wound chamber may be unharmed. In some embodiments, treatment does not result in a change to corneal epithelium, stroma, or endothelium integrity. In some embodiments, treatment does not result in a change to corneal thickness, intraocular pressure, or corneal surface morphology, i.e., no abrasion. In some embodiments, long-term vision damage may be prevented. In some embodiments, eyelid retraction and/or eyelid scarring may be reduced. In some embodiments, corneal swelling, infiltrate, or neovascularization may be avoided. In some embodiments, ocular infection may be resolved within fourteen days.

In some embodiments, the device chamber may be left in place for up to 7 days prior to replacement. In some embodiments, a therapeutic agent within the isolated treatment space may be replaced at a minimum frequency of about every 24 hours.

In further embodiments, the methods disclosed herein may include identifying the subject as having an ocular condition.

In further embodiments, the methods disclosed herein may include decontaminating and/or debriding the ocular wound.

In further embodiments, the methods disclosed herein may include imaging the eye of the subject.

In further embodiments, the methods disclosed herein may include monitoring the environment of the eye. Monitoring of the environment of the eye may include measuring at least one of a temperature, oxygen, and pH level within the isolated treatment space.

In further embodiments, the methods disclosed herein may include applying negative pressure therapy to the eye region.

In some embodiments, treatment with the ocular treatment device increases a blood plasma concentration of the therapeutic agent in the subject relative to administration of the therapeutic agent alone.

In some embodiments, the device chamber may be secured to the subject after onset of an ocular infection or an ocular injury. In some embodiments, the device chamber may be applied at a point of injury for stabilization. In some embodiments, the device chamber may be secured prophylactically.

In some embodiments, the ocular condition of the subject may be treated in conjunction with a facial wound of the subject.

### BRIEF DESCRIPTION OF THE DRAWINGS

The accompanying drawings are not intended to be drawn to scale. For purposes of clarity, not every component may be labeled in the drawings. In the drawings:

FIG. 1 illustrates an expanded view of an ocular treatment device in accordance with one or more embodiments;

FIG. 2 illustrates a top down view of the ocular treatment device illustrated in FIG. 1;

FIG. 3 illustrates a vertical cross-section of the ocular treatment device illustrated in FIG. 1;

FIG. 4 illustrates a perspective view of the ocular treatment device illustrated in FIG. 1;

FIG. 5 illustrates ocular treatment devices of varying sizes in accordance with one or more embodiments;

FIGS. 6A-6B illustrate the ocular treatment device of FIG. 1 secured over a periorbital region of a subject. FIG. 6A illustrates a front view. FIG. 6B illustrates a side profile view;

FIG. 7 illustrates a treatment device as described herein secured over a periorbital region of a subject in accordance with one or more embodiments;

FIGS. 8A-8B illustrate an embodiment of an ocular treatment device positioned over both eyes of a subject. FIG. 8A illustrates a front view. FIG. 8B illustrates a side profile view;

FIG. 9 illustrates white light images of eyes taken just after corneal injury and infection induction and at 0, 24, and 72 h post-treatment with and without an ocular treatment device in accordance with one or more embodiments;

FIGS. 10A-10B illustrate fluorescein staining eyes taken just after corneal injury and infection induction and at 0, 24, and 72 h post-treatment with and without an ocular treatment device in accordance with one or more embodiments. FIG. 10A illustrates conduction of the fluorescein staining. FIG. 10B illustrates quantitation of the fluorescein staining;

FIG. 11 illustrates representative corneal SD-OCT images of untreated, 0.5% moxifloxacin ophthalmic drops, and ocular treatment device treated eyes prior to blepharotomy, corneal abrasion and infection and immediately post corneal abrasion injury and at 0, 24, and 72 h post-treatment;

FIGS. 12A-12B illustrate colony forming unit (CFU) counts on treated ocular tissue to assess bacterial load. FIG. 12A illustrates CFU counts 24 hours post treatment. FIG. 12B illustrates CFU counts 72 hours post treatment;

FIGS. 13A-13B illustrate CFU counts on treated periocular tissue to assess bacterial load. FIG. 13A illustrates CFU counts 24 hours post treatment. FIG. 13B illustrates CFU counts 72 hours post treatment; and

FIGS. 14A-14B illustrate post treatment blood plasma samples to assess antibiotic presence. FIG. 14A illustrates plasma samples 24 hours post treatment. FIG. 14B illustrates plasma samples 72 hours post treatment.

### DETAILED DESCRIPTION

In accordance with one or more embodiments, a device for use in the treatment of various ocular conditions is disclosed. The device may generally serve as a protective dressing for an ocular structure of a subject while facilitating treatment thereof. The device may provide an isolated treatment space in which an eye region of a subject is shielded from an outer environment. In at least some embodiments, corneal protection may be provided.

In accordance with one or more embodiments, therapy for an ocular condition may be administered within the isolated treatment space. For example, various therapeutic agents including antibiotics and analgesics may be precisely introduced and/or instilled via the treatment device. The treatment device may further maintain an installed therapeutic agent at the site of an ocular condition within the isolated treatment space. The therapeutic agent may be kept in contact with the eye and/or periorbital tissue of the subject for treatment.

Beneficially, the treatment device may provide an environment conducive to ocular healing. In at least some embodiments, a moist or wet environment may be provided within the isolated treatment space to facilitate healing. The site of an ocular condition may be bathed in a therapeutic solution to promote healing thereof. An in vivo, tissue culture-like condition may generally be promoted therein. The eye and/or periorbital tissue of the subject may be treated.

In accordance with one or more embodiments, the progression of an ocular condition may be stopped and healing may be accelerated. Injures may be stabilized and treatment initiated in a rapid manner. Immediate or early-stage enclosure of an eye region of a subject with the disclosed devices may deter progression of an ocular condition. Corneal desiccation and/or corneal scarring may be prevented. Burn wound depth progression may be reduced in the moist environment provided by the disclosed devices.

In accordance with one or more embodiments, the ocular device may include a chamber configured to enclose an eye region of a subject. The eye region may span the area around one eye of a subject or may span both eyes of a subject. In some embodiments, the ocular treatment device may generally be constructed and arranged as an orbital rim. As used herein, the term eye region may refer to an eye and/or surrounding periorbital tissue of a subject, i.e., an eyelid. The treatment device may provide a removable cover for an eye region of a subject. The device may generally be consistent with ocular anatomy and supportive of its health. The device may be optimized in terms of its size and shape to provide a predetermined reservoir based on a desired volume and/or concentration of therapeutic agent to be administered as described herein in order to form a fluid-filled treatment environment.

In accordance with one or more embodiments, the ocular treatment device may include a chamber configured to enclose an eye region of a subject. The chamber may comprise an upper assembly comprising a port, a central assembly connected to a lower surface of the upper assembly, and a lower assembly connected to a lower surface of the central assembly. The lower assembly may have a longest linear dimension, e.g., a diameter, that is greater than that of the central assembly. The upper assembly, central assembly, and lower assembly together may form an interior surface defining an isolated treatment space. The ocular treatment device may further include a sealing portion at the lower assembly of the chamber configured to be secured as an orbital rim around an eye of the subject to form the isolated treatment space.

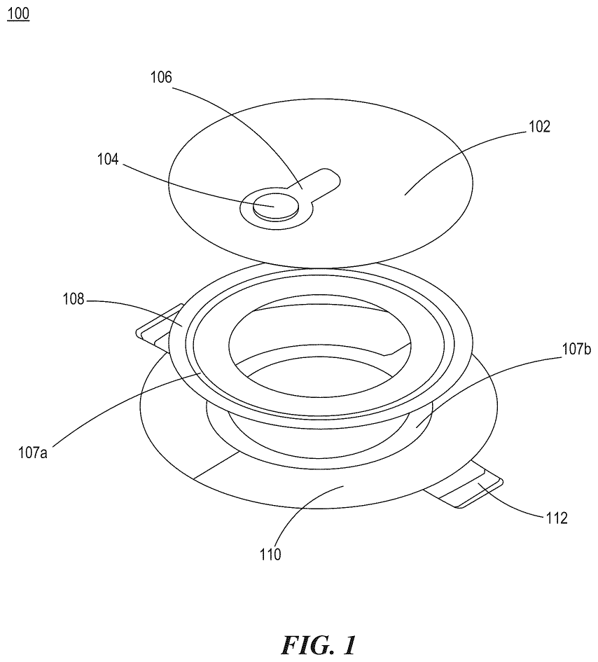

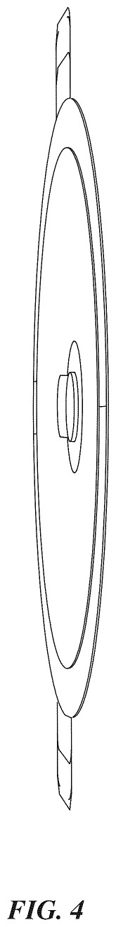

An example embodiment of an ocular treatment device is illustrated in FIGS. 1-4.

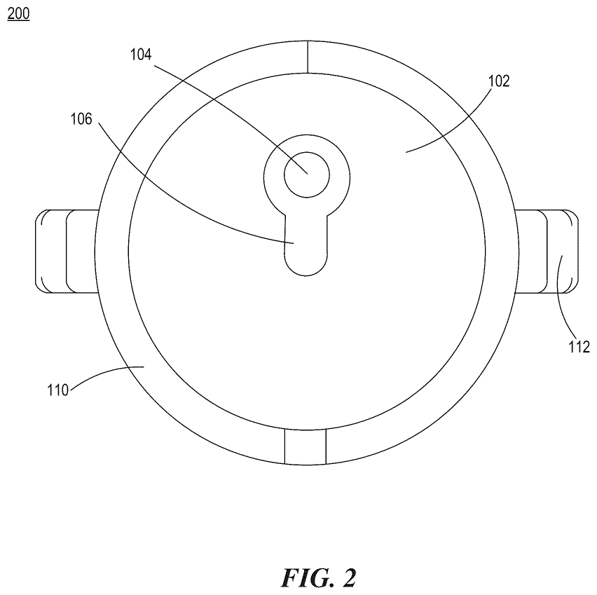

With reference to FIG. 1, an ocular treatment device 100 includes an upper assembly 102 that includes an injection port 104 that has an injection port cover 106. Injection port 104 may be a self-sealing injection port, e.g., a septum. The ocular treatment device 100 includes a central assembly 108 connected to a lower assembly 110 comprised of a permeable adhesive layer. The upper assembly 102 is connected to the central assembly 108 using seal joint 107a and the lower assembly 110 is connected to the central assembly 108 using seal joint 107b. In FIG. 1, lower assembly 110 has a longest linear dimension, e.g., a diameter, that is greater than that of the central assembly 108. The lower assembly 110 further includes a pair of application tabs 112 positioned on opposite sides of the lower assembly 110. The upper assembly 102, central assembly 108, and lower assembly 110 together form an interior surface that may define an isolated treatment space. With reference to FIG. 2, the ocular treatment device 100 has the upper assembly 102 with the injection port 104 and injection port cover 106 positioned near an edge of the upper assembly 102. The application tabs 112 of lower assembly 110 are positioned in the center of the peripheral edge of the lower assembly 110. Further shown in FIG. 2 are example dimensions of both the upper assembly 102 and lower assembly 110. For example, an upper assembly as described herein may provide for a concave ocular treatment device. The concavity may be optimized to provide for a specific volume of fluid, e.g., a therapeutic, to be placed within the ocular treatment device.

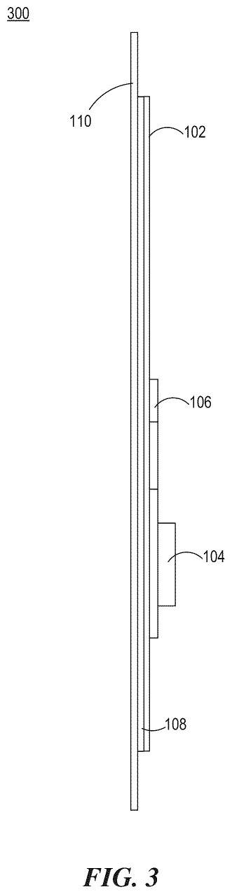

With reference to FIG. 3, which illustrates a vertical cross-section of the ocular treatment device of FIG. 1, the ocular treatment device 300 includes upper assembly 102 with injection port 104 and injection port cover 106. Central assembly 108 is connected to the upper assembly 102 and lower assembly 110. As noted herein, lower assembly 110 has a largest linear dimension, e.g., a diameter, that is larger than that of the central assembly 108. In the configuration shown, the connection between the larger lower assembly 110 and smaller central assembly 108 can form a bellows shape that allows for liquids, such as therapeutics, saline, or buffered solutions, to collect in the volume defined by the shape of the bellows and thus maintain proximity to the eye during use of the ocular treatment device. The bellows shape of central assembly 108 may further prevent additional fluid pooling in the space between the central assembly 108 and lower assembly 110 that is positioned further from the eye of a subject when the ocular treatment device is positioned.

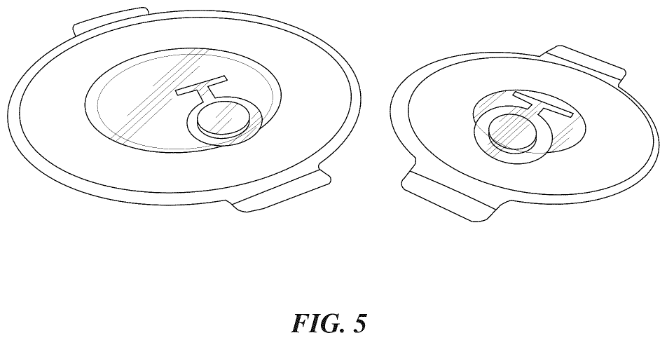

The overall geometry of the ocular treatment device may also be optimized for suitability in terms of use over one or both eyes of a subject, i.e., to cover a desired portion of the eyes and periorbital tissue of the subject. For example, a single eye and eyelid may be encompassed by a substantially circular or elliptical device. In such specific non-limiting embodiments, the device may be about 1 to about 4 inches in diameter. Devices of various diameters are presented in FIG. 5. In other non-limiting embodiments, a substantially oblong device, for example, about 3 by about 6 inches may be implemented in order to cover both eyes and accompanying periorbital tissue of the subject. In some embodiments, the device may have a shape that substantially follows the curvature and dimensions of an ocular region of a human anatomy. For example, the device may have an overall shape that conforms to at least one of the various regions of human facial anatomy, such as the orbital/periorbital region immediately surrounding the eye, the frontal region, i.e., the forehead, the infraorbital and/or zygomatic regions below the eye, i.e., the cheekbones, and the nasal region, i.e., the bridge of the nose. The curvature of the ocular treatment device may be shaped directedly into the device material. Alternatively, the device material may be sufficiently flexible to allow it to follow the contours of the aforementioned anatomical regions.

In accordance with one or more embodiments, the chamber may include an interior surface that defines an isolated treatment space. The isolated treatment space may be configured to immerse the eye and/or periorbital tissue of the subject in a therapeutic agent. The chamber and defined isolated treatment space may be configured to protect and/or prevent ocular injury. The chamber may be made of any material consistent with its use as a vehicle for therapeutic delivery. For example, if a liquid or gel is the mode of therapeutic administration, then a polyurethane material might be selected for the chamber. The chamber may be constructed of a material that promotes safe oxygen transmissibility across a cornea of a subject. A material with a predetermined oxygen permeability level may be selected depending on need. Biocompatibility may also be a significant consideration. Requirements specific to ocular indications, for example, limits on ethylene oxide residuals may also be a design consideration. In some embodiments, the chamber may be made of a substantially conformable material. A substantially transparent or semi-transparent material may be used to promote visibility by both the subject and clinician. The chamber may be constructed of a material that is treated, i.e., UV treated depending on intended application. In at least some embodiments, the chamber may be made of a single sheet of material. In some embodiments, the chamber may be characterized by a bellows structure.

In accordance with one or more embodiments, the device may include a sealing portion at the base of the chamber. The sealing portion may be configured to be secured as an orbital rim around the eye of the subject to form the isolated treatment space. The sealing portion may be an adhesive or mechanical seal. The sealing portion may be configured to adhere to compromised tissue, i.e., burned tissue. The adhesive may be made of a biocompatible material. For example, the adhesive may be breathable. In at least some non-limiting embodiments, an acrylic adhesive may be used. In accordance with one or more embodiments, tabs may facilitate attachment and removal of the device from a subject. The seal should also be consistent with use as a vehicle for therapeutic delivery in terms of providing a liquid-tight seal. In this way, ocular tissue may remain bathed in therapeutic agent, rather than only keep the tissue moist.

In accordance with one or more embodiments, the device may include a port to facilitate material transfer into and/or out of the chamber. Oxygen may be introduced via the port. This may generally augment or supplement the oxygen transmission characteristics of the chamber material. Therapeutic agents may also be introduced as described below in connection with various methods of treatment. Precise delivery at specific volumes and/or concentration is achievable. The port may facilitate the provision of a fluid-filled treatment environment. The port may be sealable and, in some non-limiting embodiments, may be self-scaling. Various reagents and/or therapeutic agents may be injected into the chamber via the self-scaling port. In some embodiments, a tube may cooperate with the port. The tube may be connected to the port and in fluid communication with the isolated treatment space. The tube may facilitate removing fluid from or introducing fluid to the isolated treatment space.

In accordance with one or more embodiments, the port and/or tube may be configured to enable the provision of negative pressure therapy within the isolated treatment space. In contrast to conventional negative pressure treatment chambers, the chamber of the disclosed ocular treatment device does not contain a porous insert.

In accordance with one or more embodiments, an ocular treatment device may be integrated into an overall facial treatment device, i.e., a facial wound chamber. The device may be configured for use in conjunction with a protective shield.

In accordance with one or more embodiments, a base may be implemented in cooperation with the sealing portion of the ocular treatment device to provide additional elevation as may be required, for example, to accommodate surface anatomy of a subject. The base may be of a different color than the rest of the device.

In accordance with one or more embodiments, the interior surface of the chamber may be characterized by a plurality of embossed structures. In other embodiments, the interior surface of the chamber may be unembossed. Such structures may be configured to directly contact periorbital tissue, i.e., the eyelid of the subject. These embossed structures may be configured in a way to create pathways between the interior surface of the chamber and the eye region of the subject. Such pathways may facilitate negative pressure therapy. In some embodiments, the structures may be semi-rigid. The embossed structures may be positioned in a uniform pattern on the interior surface of the chamber. The embossed structures may be positioned at varying distances from one another. In some non-limiting embodiments, for example, separation therebetween may be about 0.2 mm to about 10 mm apart. These embossed structures may also be of varying height, for example, they may have a height at a minimum of about 0.1 mm to a maximum of 5 mm in some non-limiting embodiments. The embossed structures can be of various geometry, for example, they may be shaped as cones, pyramids, pentagons, hexagons, half-spheres, domes, rods, elongated ridges with round sides, or elongated ridges with square sides. In some embodiments, the embossed structures may cover at least about 50% to about 100% of the interior surface of the chamber.

In accordance with one or more embodiments, the ocular treatment device may have no detrimental impact on periocular tissue surrounding the orbital rim to which the device is adhered. There should also be no adverse effects under the adhesive rim. The device may adequately adhere to compromised tissue, e.g., burned tissue. The device may not have a deleterious effect on corneal integrity. The device may not interfere with safe oxygen transmissibility levels across the cornea. The device adherent to the periorbital rim may be safely installed on skin micrografts, split-thickness skin grafts, and cultured autologous keratinocytes. Corneal thickness and corneal surface features should not exhibit any significant changes. No significant change in intraocular pressure should occur. No adverse effect in connection with an ocular condition. No inflammation, infiltrate, or neovascularization should be associated with use of the device.

In accordance with one or more embodiments, various sensors may be associated with the ocular chamber. For example, monitoring oxygen levels may be of importance in connection with the cornea in view of it being a superficial tissue layer with no dedicated blood supply. The device may include one or more sensors to measure and/or monitor an oxygen, temperature, pH, bacterial, or other level within the isolated treatment space. In accordance with one or more embodiments, a temperature sensor may comprise a plurality of filamentary structures disposed onto a flexible substrate. For example, a temperature sensor may include a plurality of metallic filamentary structures, such as gold filaments, disposed on a flexible substrate. In accordance with one or more embodiments, an oxygen sensor may comprise at least one light source and a photodetector disposed onto a flexible substrate. For example, an oxygen sensor may comprise a light emitting diode (LED) that emits in the visible or infrared region of the electromagnetic spectrum. In accordance with one or more embodiments, a pH sensor may comprise at least one pH-sensitive dye incorporated into a polymer membrane and a photodetector. In any embodiment of a sensor, the sensor may be configured to send collected data over a suitable wireless signal, for example, over a cellular network, BLUETOOTH® wireless data transmission protocol, or other wireless data transmission protocol known in the art, to a receiver, such as a cellular phone, tablet, computer, server, or the like.

In accordance with one or more embodiments, the device may also include one or more auxiliary components such as but not limited to a fluid trap, a pump, an exhaust port, and a suction device.

In accordance with one or more embodiments, a kit may include an ocular treatment device as described herein, alone or in conjunction with a source of a therapeutic agent and/or one or more sensors and auxiliary components as described herein. The kit may also include instructions for performing a method of treating various ocular conditions as described herein.

In accordance with one or more embodiments, the ocular device may be configured for single use, e.g., disposable. In some other embodiments, the ocular device may be removable and/or rescalable. The device may be provided in substantially sterile packaging.

In accordance with one or more embodiments, various ocular conditions may be treated with the disclosed devices. In some embodiments, ocular infections may be treated. For example, bacterial or viral keratitis, i.e., Staphylococcus aureus, Streptococcus spp., and Pseudomonas aeruginosa-induced keratitis may be treated. In accordance with one or more other embodiments, ocular wounds or injuries may be treated. In some embodiments, an ocular wound, sore, laceration, abrasion, or surgical incision may be treated. In some embodiments, a blepharoplasty site may be treated. Damaged or compromised periorbital tissue or structure may be treated. In some specific embodiments, exposure keratopathy may be treated. In another specific embodiment, ocular burns such as those associated with facial burns may be treated. In at least some embodiments, the ocular condition may pertain to the cornea. For example, in yet another specific embodiment, corneal ulcers or corneal epithelial wounds are treated. In some embodiments, the ocular condition may relate to periorbital tissue, i.e., eyelid injury.

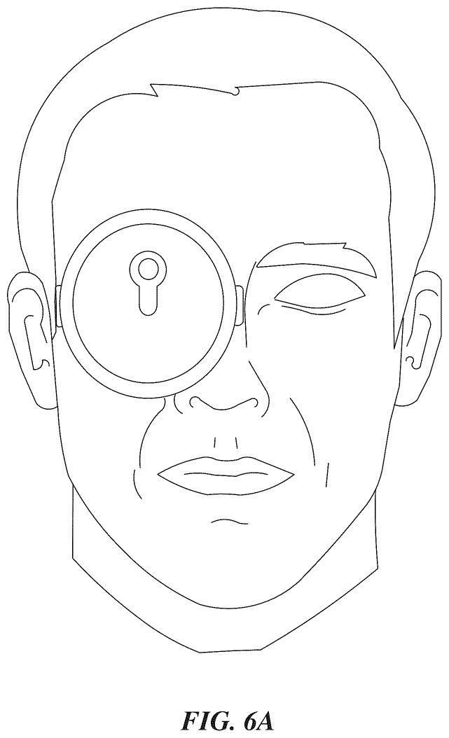

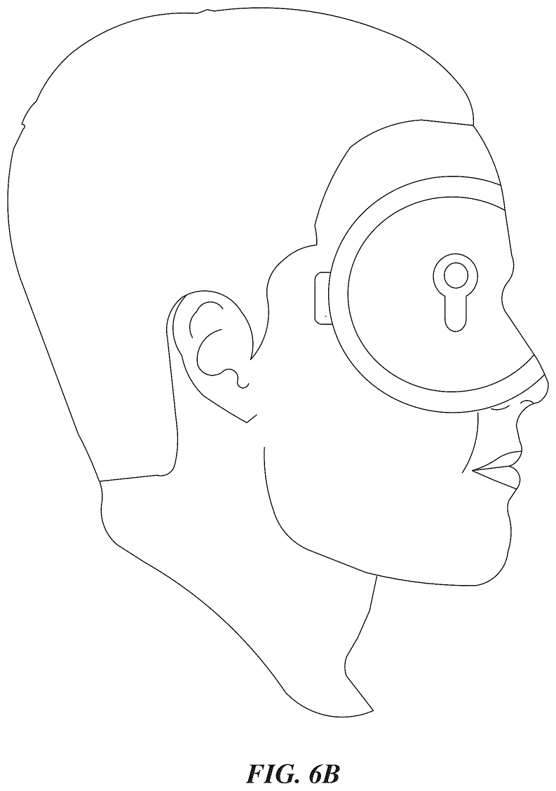

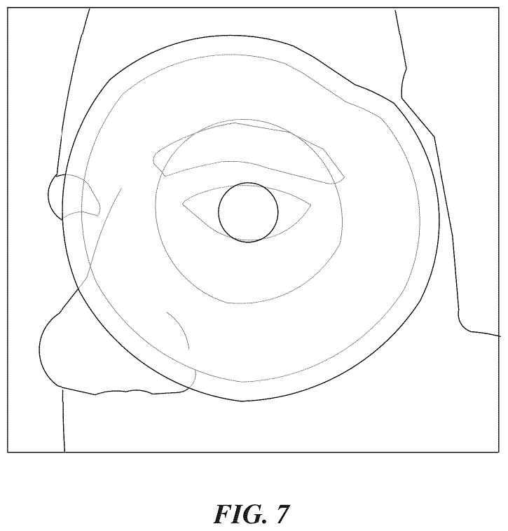

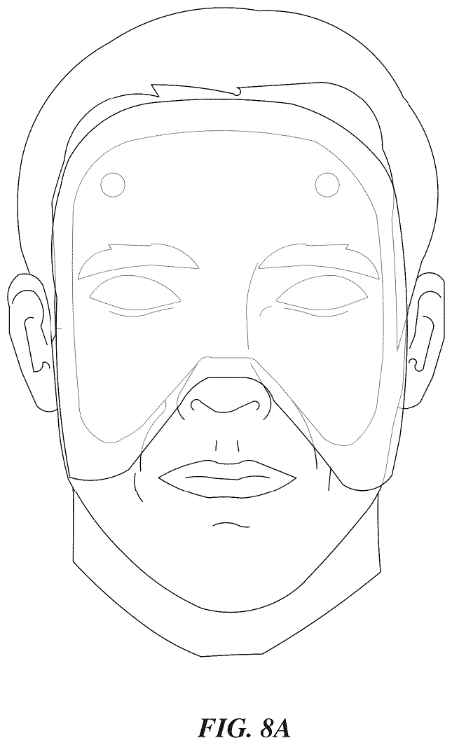

In accordance with one or more embodiments, a method of treating an ocular condition, such as a method of treating bacterial keratitis, may involve securing an ocular treatment device as described herein over a periorbital region of a subject as presented in FIGS. 6A, 6B, 7, 8A, and 8B. For example, an ocular treatment device as described herein may be secured over one eye of a subject as illustrated in FIGS. 6A, 6B, and 7. Alternatively, an ocular treatment device as described herein may be secured over both eyes of a subject as illustrated in FIGS. 8A and 8B. Beneficially, the device may be safely and effectively applied as a periorbital rim even if the subject has a compromised periocular structure, such as one associated with significant ocular exposure. A skin graft, i.e., partial or full thickness skin graft, or other technique may be used to augment and/or reestablish normal lid coverage. Beneficially, detrimental contraction conventionally associated with grafting may be lessened or avoided. A subject may be identified as having an ocular condition requiring treatment. Prior to or concurrent with application of the device, the eye region of the subject may be imaged. The site for ocular therapy may be decontaminated and/or debrided. The ocular device may be secured to a subject shortly after onset of an ocular infection or injury. Alternatively, the ocular device may be secured prophylactically so as to prevent infections at a point of injury thus providing an advantage in hastening injury repair and treatment. Beneficially, the ocular device may be applied at a point of injury for stabilization.

In accordance with one or more embodiments, a therapeutic agent may be administered to the isolated treatment space of the device. Any approved therapeutic agent may be introduced. A therapeutic agent may be formulated for administration to the ocular chamber. For example, the therapeutic agent may be administered as a liquid, suspension, gel, hydrogel, ointment, or foam. In some embodiments, an antimicrobial agent may be introduced to the ocular chamber. Antibiotics, analgesics, anti-inflammatory, antifungal, various molecules, growth factors, cells, e.g., stem cells or micrographs, and other therapies can be delivered. In some non-limiting embodiments, vancomycin may be delivered. In other non-limiting embodiments, moxifloxacin may be delivered. Other reagents such as medium or saline may be added. In accordance with one or more embodiments, the therapeutic agent may be formulated for sustained release.

In accordance with one or more embodiments, such as in a method of treating an ocular condition, high concentrations of therapeutic agents, e.g., about 10 to about 1000 times the minimum inhibitory concentration (MCI), may be introduced to an eye of a subject for treatment of an ocular condition. In some embodiments, antibiotic concentrations up to about 100 times MIC may be used. In other embodiments, antibiotic concentrations of at least about 100 times MIC may be used.

In accordance with one or more embodiments, a wet or moist environment in the isolated treatment space may be promoted. A target portion of the eye region, i.e., eye, cornea, eyelid, or other periorbital tissue may be immersed in a fluid environment within the isolated treatment space to promote healing via substantially continuous contact. In some nonlimiting embodiments, the ocular device may be replaced periodically, for example, about every 72 hours or up to about every 7 days. Likewise, in some non-limiting embodiments, the therapeutic agent may be replaced periodically, i.e., about every 24 hours. Oxygen may be administered as described above. Negative pressure therapy may also be administered to the isolated treatment space.

In accordance with one or more embodiments, an environment of the eye region of the subject may be monitored. For example, a temperature, oxygen, carbon dioxide, and/or PH level within the isolated treatment space may be monitored.

In accordance with one or more embodiments, a single eye and/or associated periorbital tissue of a subject may be treated. In other embodiments, both eyes and/or associated periorbital tissue of a subject may be treated. In at least some embodiments, ocular treatment may be performed in conjunction with treatment of a facial wound of the subject.

In accordance with one or more embodiments, a bacterial load associated with an ocular condition may be decreased. No or minimal inflammation may be exhibited. Burn wound necrosis thickness may be decreased. Scarring and lid retraction may be reduced. In accordance with one or more embodiments, ocular treatment may improve corneal integrity, reduce corneal desiccation, and/or reduce corneal scarring. Ocular treatment in accordance with one or more embodiments may slow or prevent progression of ocular wound depth. Surrounding tissue and/or tissue under an adhesive rim of the ocular device may be unharmed. Treatment does not result in a significant change to corneal epithelium, stroma, or endothelium integrity. Treatment does not result in a significant change to corneal thickness, intraocular pressure, or corneal surface morphology, i.e., no abrasion. Long-term vision damage may be prevented. Eyelid retraction and/or eyelid scarring may be reduced. Corneal swelling, infiltrate, and neovascularization may be avoided. In at least some embodiments, an ocular infection may beneficially be resolved within fourteen days or earlier. In at least some embodiments a corneal epithelial defect may heal in as quickly as 48 hours.

In accordance with various embodiments, devices and methods may involve one or more aspects as disclosed in International (PCT) Patent Application Publication No. WO 2018/119442 to Eriksson et al. which is hereby incorporated herein by reference in its entirety for all purposes.

### EXAMPLES

The function and advantages of these and other embodiments can be better understood from the following examples. These examples are intended to be illustrative in nature and are not considered to be in any way limiting the scope of the invention.

To illustrate the efficacy of the ocular treatment device as disclosed herein, the following example describes experiments where bacterial infections were inoculated onto the cornea of guinea pigs and treated both with and without an ocular treatment device.

### Animals

All female Institute Armand Frappier (IAF) hairless, guinea pigs (200-250 g, Crl: HA-Hrhr) were acquired from Charles River Laboratories (Wilmington, MA) and acclimated for at least 72 h prior to the beginning of the study. Randomly grouped animals (N=4 per group) were anesthetized and administered analgesics. Research was conducted in compliance with the Animal Welfare Act, the implementing Animal Welfare Regulations, and the principles of the Guide for the Care and Use of Laboratory Animals from the National Research Council. The facility's Institutional Animal Care and Use Committee (IACUC) approved all research conducted in this study. The facility where this research was conducted is fully accredited by AAALAC. All animals were maintained and handled according to institutional guidelines and the ARVO Statement for the Use of Animals in Ophthalmic and Vision Research.

### Blepharotomy

A blepharotomy was performed to remove the upper and lower eyelids. Briefly, after general anesthetization of animals, a 25-gauge needle was used to inject 0.05-0.1 mL of 2% lidocaine HCL with epinephrine 1:100,000 USP into the upper and lower eyelids to control for excess bleeding. Forceps with teeth (#12) were used to grip the upper and lower eyelids while Westcott scissors were employed to remove the entire upper and lower lid from the orbital rim. Hemostasis was controlled with a high temperature handheld cautery device as necessary. Animals were allowed to recover from the blepharotomy procedure for 72 h prior to corneal epithelial wound creation and infection induction as described herein.

### Pseudomonas aeruginosa Isolates

P. aeruginosa strain 27853 was obtained from American Type Culture Collection (ATCC, Manassas, VA). Stock cultures were initially grown in Luria Bertani (LB) broth (Lennox) microbial growth medium (Sigma-Aldrich, St. Louis, MO) overnight at 37° C. with shaking. For bacterial colony isolation, a 1 μL inoculation loop was used to inoculate an ALFA AESAR™ LB Agar plate, plain (Fisher Scientific, Waltham, MA). After overnight incubation at 37° C., a colony with a small morphology, shown to produce both fluorescein and pyocyanin, was isolated and subcultured on Pseudomonas isolation agar (Hardy Diagnostics, Santa Maria, CA). For experimental studies, a bacterial culture was grown overnight at 37° C. with shaking in 5 mL LB broth from an isolated colony cultured on Pseudomonas isolation agar (Hardy Diagnostics, Santa Maria, CA). Spectrophotometric readings (520 nm; SMARTSPEC™ PLUS Spectrophotometer, BioRad, Hercules, CA) were taken of overnight cultures followed by dilution to a bacterial concentration of 1×10⁸ CFU/mL. Bacterial dilutions were serially plated to confirm the CFU/mL placed on the eye.

### P. aeruginosa Keratitis Model

Seventy-two hours after the blepharotomy procedure, a corneal epithelial wound was created using a corneal rust ring remover (ALGERBRUSH® II, Alger Company, Inc., Lago Vista, TX) immediately prior to the introduction of P. aeruginosa 27853. Briefly, a trephine was used to demarcate a 4 mm area on the central cornea of left eyes followed by epithelium removal. Fluorescein staining was used to verify the uniform removal of the epithelium. Following creation of the corneal epithelial defect, 1×10⁸ CFU/mL of P. aeruginosa 27853 was applied to the left of eye of each guinea pig. Animals were allowed to recover for 24 h prior to commencement of treatment. After 24 h, treatment animals received an ocular treatment device loaded with 500 μL of 0.5% moxifloxacin ophthalmic solution (Alcon Fort Worth, TX) (N=4) or were administered a 10 μL drop of 0.5% moxifloxacin ophthalmic solution (N=4) to the infected eye four times daily. For animals that received an ocular treatment device, the eye and surrounding tissues were prepared as follows. Following the removal of residual periorbital hair using a depilatory cream, the skin was cleaned with 70% alcohol and dried to ensure adherence of the ocular treatment device. After application of the ocular treatment device, 500 μL of 0.5% moxifloxacin ophthalmic solution was injected into the ocular treatment device through the self-sealing silicone port using a 25-gauge 1″ needle. The ocular treatment device and 0.5% moxifloxacin was replaced every 24 h after assessments and imaging for the duration of the study.

### Bacterial Quantification

After humane euthanization of animals, whole eye globes were removed and immediately placed on ice. Samples were placed in sterile tubes containing ceramic beads (MAGNA LYSER® Green Beads, Roche Indianapolis, IN, USA) and 1 mL sterile phosphate buffered saline (PBS; Gibco, Grand Island, NY). The samples were homogenized for 60 s at 5.5 m/s utilizing a bead beater (MP BIOMEDICALS FASTPREP-24™5G Instrument, Santa Ana, California). Following homogenization, the homogenate was plated at 1/10 and 1/100 dilutions on Pseudomonas isolation agar (Hardy Diagnostics). Plates were allowed to dry before being inverted and placed at 37° C. overnight. After overnight incubation, colonies were manually counted. The CFU/mL was calculated using the formula: (number of colonies X dilution factor)/volume of culture plate.

### White Light and Fluorescein Ocular Imaging

White light and fluorescein ocular imaging was performed. Images were collected at specified time points using a surgical microscope equipped with a camera and a cobalt blue filter (OPMI VISU 200 S8; Carl Zeiss Surgical, Oberkochen, Germany). Sterile fluorescein sodium ophthalmic films USP (FLUORETS®, Chauvin Laboratory, Aubenas, France) were moistened with 100 μL of balanced salt solution (BSS; Alcon, Fort Worth, TX) and a drop was placed onto the eye for 10 s before the eyes were rinsed with BSS (Alcon). Images of fluorescein uptake were acquired using a surgical microscope under cobalt blue light and analysis was performed using ImageJ (NIH, Bethesda, MD). The data is reported as the area of fluorescein uptake in pixels.

### Optical Coherence Tomography

Images of infected eyes were obtained to visualize corneal surface anatomy using spectral domain optical coherence tomography (SD-OCT, Micron IV; Phoenix Research Laboratories, Pleasanton, CA) at the indicated time points.

### Liquid Chromatography-Tandem Mass Spectrometry

Plasma samples were thawed on ice for 30 min and subsequently centrifuged at 17,000 g for 20 min. Supernatants were spiked with internal standard vancomycin (Sigma-Aldrich) at 1 μg/mL and stored at −70° until liquid chromatography-mass spectrometry-mass spectrometry (LC-MS-MS) analysis was performed. LC/MS-MS analysis was conducted by binding antibiotics to a 2.1×50 mm, 5 μm C18 column (Agilent ZORBAX® Eclipse XDB 80 Å, Agilent Technologies, Santa Clara, CA) at 40° C. for 30 s with 100% mobile phase A. The column was eluted with a linear gradient from 0% to 90% mobile phase B for 90 s. Analysis time was 4 m for one injection. Moxifloxacin (Sigma-Aldrich) standards of 0-2 μg/mL were used to generate a standard curve. Vancomycin (1 μg/mL, Sigma-Aldrich) served as the internal control.

Mass spectrometric analysis was carried out on an AB Sciex API 4000 mass spectrometer (AB Sciex, Framingham, MA) with an electrospray ionization (ESI) interface and triple quadrupole mass analyzer. The mass spectrometer was operated in multiple reaction monitoring mode via the positive electrospray ionization interface using the mass-to-charge ratios (m/z) of 725.5/144.0 (vancomycin) and 402.2/358.1 (moxifloxacin). For the MS/MS analysis of vancomycin, the declustering potential was set to 75 V, the collision energy was set to 24 V, and the collision cell exit potential was set to 7.2 V with an m/z set at 725.5/144.0. For the MS-MS analysis of moxifloxacin, the declustering potential was set to 90 V, the collision energy set to 26.89 V, and collision cell exit potential set to 7.9 V for an m/z set at 402.2/358.1. Electrospray capillary voltage was set to 4.5 kV.

### Statistical Analyses

For fluorescein data analysis, a linear mixed model for repeated measures was used.

In each regression model, time, treatment group, and the interaction of these two factors were used as fixed explanatory variables. The area measured at the time of injury/infection was also included in the model as a separate covariate. The best fitting residual covariance structure for each outcome was determined using Akaike Information Criterion (AIC) and Bayesian Information Criterion (BIC) statistics and an autoregressive first order covariance matrix was used for fluorescein area. Fluorescein area data are presented as means and 95% confidence intervals (95% CI). Differences among treatment groups in the number of CFUs from harvested eye and skin tissue as well as the concentration of moxifloxacin were analyzed using the Kruskal-Wallis test, stratified by time point. The p-values for all post hoc pairwise comparisons (padj) were adjusted according to the appropriate method (i.e., Dwass-Steel-Critchlow-Fligner or Tukey-Kramer). CFU and moxifloxacin data are presented as medians and interquartile ranges (IQR). Significance was evaluated using an alpha of 0.05 for all studies. All analyses were performed using SAS 9.4 (SAS Institute, Cary, NC).

### Clinical Evaluation of Infected Ocular and Periocular Tissues

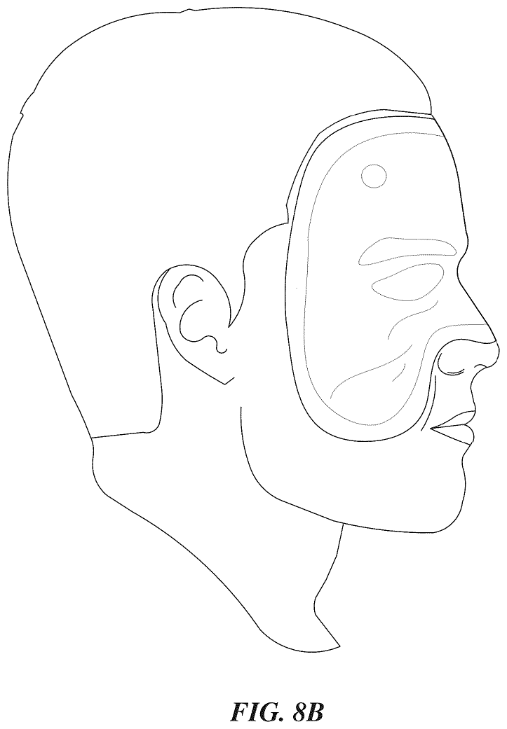

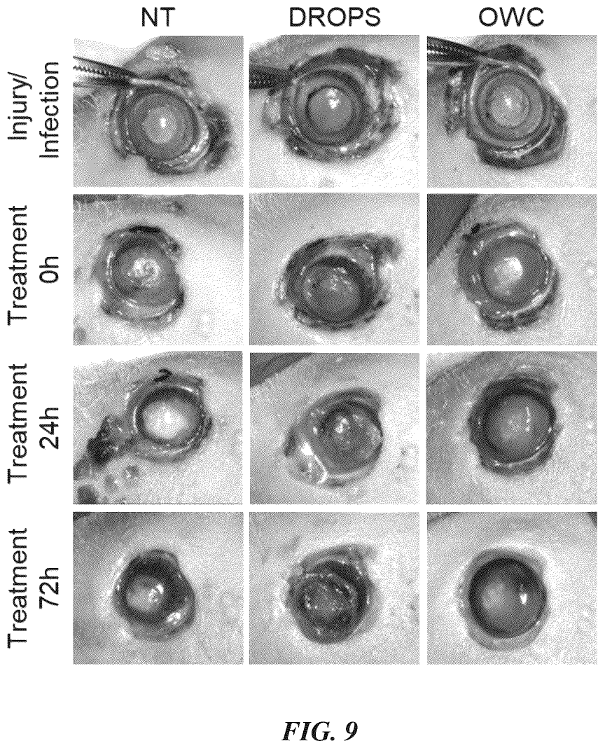

As illustrated in FIG. 9, clinical evaluation of white light imaging data qualitatively revealed less inflammation in injured periocular tissues in animals treated with the ocular treatment device when compared to those that received no treatment (NT) or were treated with 0.5% moxifloxacin ophthalmic drops at all observed time points. Further, the corneal surfaces of eyes treated with drops or NT were more desiccated than eyes treated with the ocular treatment device. While results are qualitative, these clinically observed findings are supported by previously published pathological results showing significantly lower levels inflammatory cytokines as well as fibrosis in the skin samples taken from ocular treatment device treated animals.

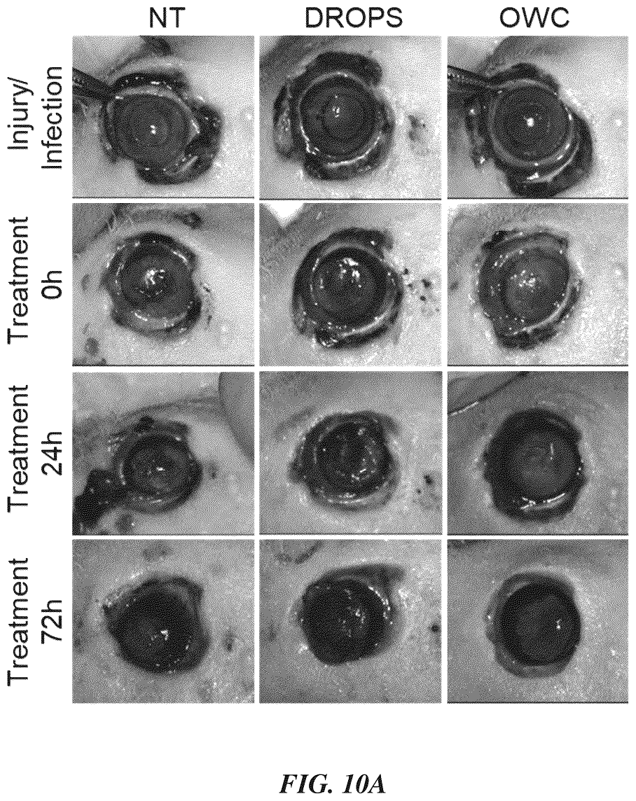

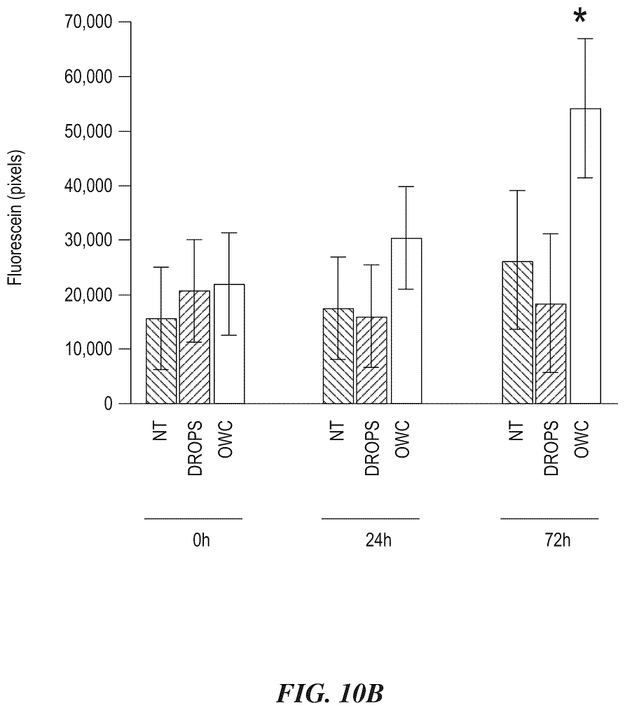

In order to assess the corneal anatomy and integrity of the corneal surface, fluorescein staining was conducted (FIG. 10A) and quantitated (FIG. 10B) at the time of inoculation with P. aeruginosa 27853 and at 0, 24 and 72 h post-treatment. The results showed significantly more fluorescein staining (p<0.05) at 72 h in eyes treated with an ocular treatment device when compared to eyes that were untreated or received 0.5% moxifloxacin ophthalmic drops

(FIGS. 10A, 10B). These data demonstrate a corneal surface that is non-desiccated and capable of fluorescein staining uptake. Indirectly, the results suggest a healthier ocular surface structure when compared to the desiccated corneas observed in the NT and 0.5% moxifloxacin ophthalmic drop groups.

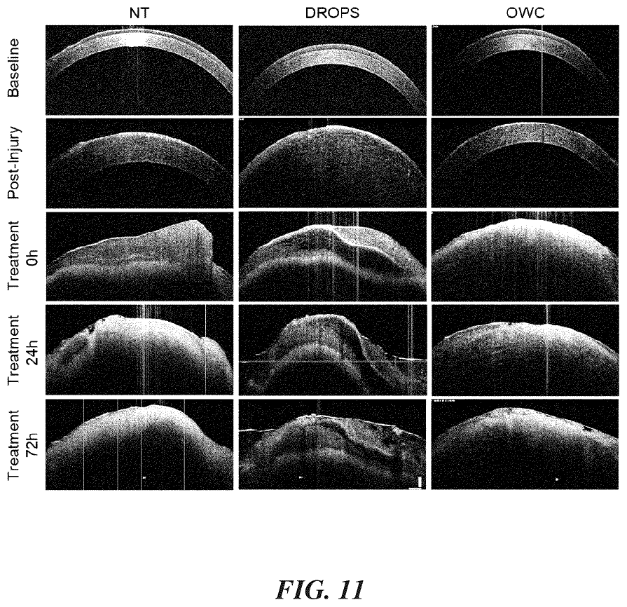

To further assess the integrity of the corneal surface anatomy, OCT imaging was conducted as shown in FIG. 11. OCT imaging results were found to support fluorescein staining observations and showed irregular corneal anatomy in NT and 0.5% moxifloxacin ophthalmic drops groups. In eyes that were treated with an ocular treatment device, some stromal edema was noted, but otherwise a more normal corneal anatomy was observed.

### Evaluation of Bacterial Load in Ocular and Periocular Tissues

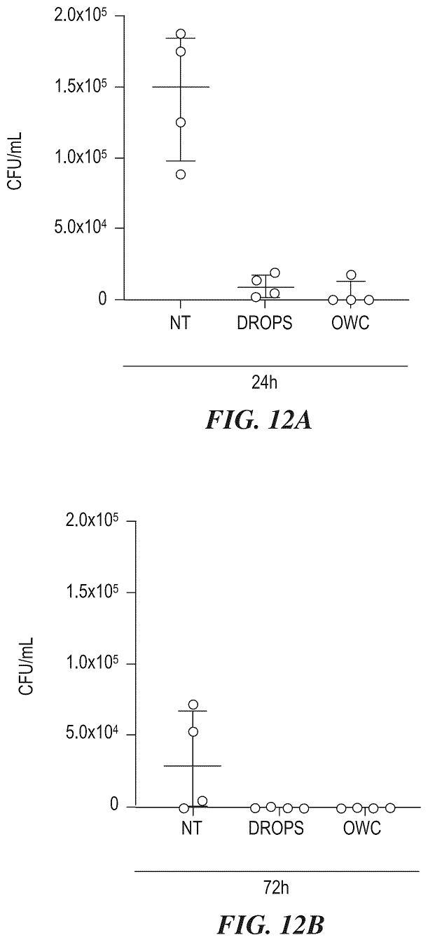

This example was intended to determine if the ocular treatment device could be used to decrease the bacterial bioburden in both the ocular and periocular tissues. Whole eye globes were harvested from animals at 24 or 72 h post-treatment and bacterial quantification was performed. As illustrated in FIG. 12A, there was a significant (p=0.01) decrease in the number of CFUs present in ocular tissues at 24 h in groups treated with either an ocular treatment device or 0.5% moxifloxacin ophthalmic drops when compared to the no treatment group.

Although there were no significant differences in the CFU counts from the ocular tissue samples between the animals that received an ocular treatment device and those that received drops at either 24 or 72 h (FIGS. 12A, 12B), there were clinically meaningful differences in the number of animals with active infections among the treatment groups. At 24 h, all the animals in the NT and 0.5% moxifloxacin ophthalmic drops groups maintained Pseudomonas infections while only 25% of the animals treated with an ocular treatment device showed infection. By 72 h, 100% of the animals that received no treatment were still infected and only 25% of animals in both 0.5% moxifloxacin ophthalmic drops and ocular treatment device groups were infected.

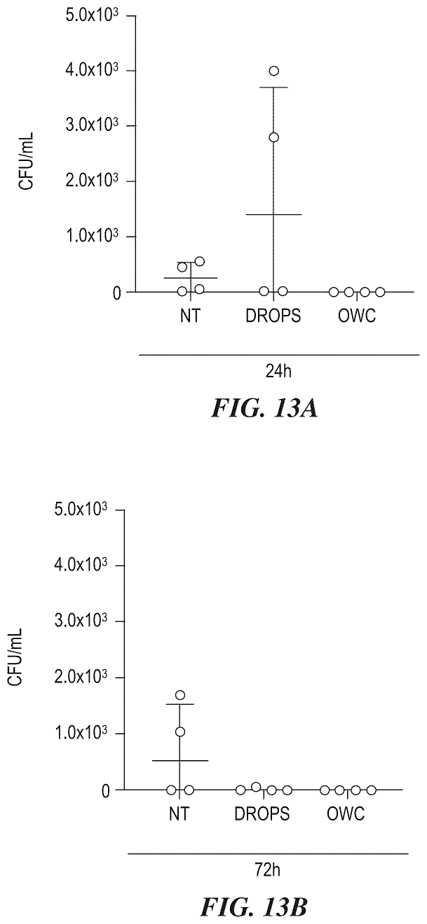

Statistical differences in CFUs were not observed among the three different treatment groups for skin tissue harvested at either time point (24 h, p=0.17; 72 h, p=0.24) (FIGS. 13A, 13B). From a clinical perspective, however, at 24 h it is noted that 50% of skin samples taken from animals that received drops and 75% of animals that received no treatment showed continued microbial infection compared to 0% of animals receiving an ocular treatment device. At 72 h, all animals treated with an ocular treatment device or 0.5% moxifloxacin ophthalmic drops showed complete microbial clearance, while 50% of those that received no treatment were still infected.

### Evaluation of Therapeutic Antimicrobial Plasma Concentrations

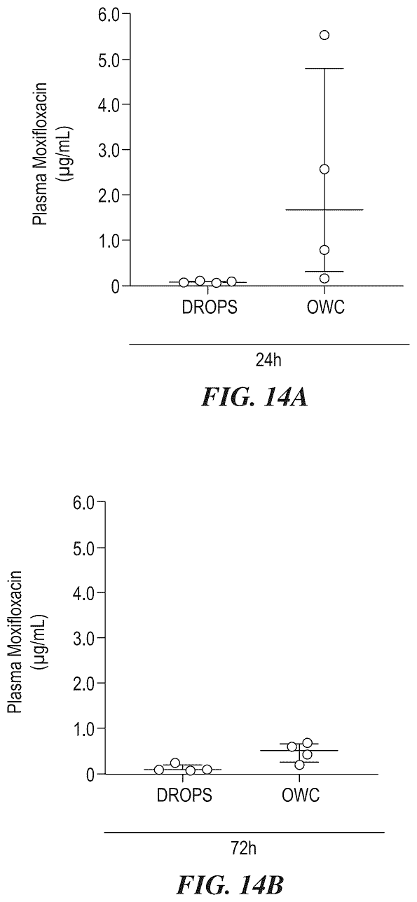

FIGS. 14A and 14B illustrate a comparison of the concentration of moxifloxacin in plasma samples taken from groups that received drops and those that received an ocular treatment device. These results have potentially meaningful clinical implications at both time points 24 (p=0.06) or 72 h (p=0.12). At 24 h post-treatment, the median plasma moxifloxacin concentration for animals treated with an ocular treatment device (median=1.68, IQR=0.47-4.05) was 24 times higher than for animals that received drops (median=0.07, IQR=0.06-0.09). By 72 h post-treatment, the median moxifloxacin plasma concentration for animals treated with an ocular treatment device (median=0.50, IQR=0.30-0.63) was approximately seven times higher than the median concentration for those that received drops (median=0.07, IQR=0.05-0.15).

It is recognized that there are limitations for using fluorescein staining as an indicator of corneal epithelial integrity in this animal model due to the extreme corneal desiccation experienced as a result of blepharotomy-induced exposure keratopathy, particularly in the NT and the standard-of-care (that is, drops only) animal groups. However, relevant clinical findings can still be gleaned from observations made during this experiment. For example, fluorescein staining is typically used to identify defects in the ocular surface and uptake is restricted in healthy eyes. In previous studies, fluorescein staining was used as a means to monitor corneal epithelial wound closure over time; as fluorescein uptake decreases, wound closure increases. Conversely, it was found that due to the desiccation of the eyes, an increase in fluorescein uptake appeared to signal an increase in eye health in those animals treated with the ocular treatment device as compared to NT or drop only simply because the desiccated eyes were unable to take up the fluorescein dye. Therefore, given the ability of the ocular treatment device to provide a fluid-filled environment to the eyes and prevent desiccation, the resulting corneal structure appeared to be more normal overall with less edema and irregular anatomy than that seen in the NT and drop only groups. Taken together, gross white light observations, fluorescein staining, and OCT imaging all suggest that eyes treated with an ocular treatment device are qualitatively healthier than those treated with drops only or not treated.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim clement does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only. Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. An ocular treatment device, comprising:
a chamber configured to enclose a region spanning both eyes of a subject, the chamber having an interior surface defining an isolated treatment space;
a sealing portion at a base of the chamber configured to be secured at the periphery of the chamber around the region spanning both eyes of the subject form the isolated treatment space; and
a port in the chamber configured to enable fluid communication with the isolated treatment space.

2. An ocular treatment device, comprising:
a chamber configured to enclose an eye region of a subject, the chamber comprising:
an upper assembly comprising a port;
a central assembly connected to a lower surface of the upper assembly; and
a lower assembly connected to a lower surface of the central assembly, the lower assembly having a longest linear dimension that is greater than that of the central assembly, the upper assembly, central assembly, and lower assembly forming an interior surface defining an isolated treatment space; and
a sealing portion at the lower assembly of the chamber configured to be secured as an orbital rim around an eye of the subject to form the isolated treatment space.

3. The ocular treatment device of claim 2, the port in the chamber is configured to enable fluid communication with the isolated treatment space.

4. The ocular treatment device of claim 3, wherein the central assembly is configured to contain a volume of a liquid delivered through the port.

5. The ocular treatment device of claim 2, wherein the eye region comprises an eye and eyelid of the subject.

6. The ocular treatment device of any of the preceding claims, further comprising a tube connected to the port and in fluid communication with the isolated treatment space.

7. The ocular treatment device of any of the preceding claims, wherein the tube is configured to remove fluid from, or introduce fluid to, the isolated treatment space.

8. The ocular treatment device of any of the preceding claims, wherein the port is further configured to enable negative pressure therapy within the isolated treatment space.

9. The ocular treatment device of any of the preceding claims, wherein the interior surface of the chamber is characterized by a plurality of embossed structures.

10. The ocular treatment device of any of the preceding claims, wherein the embossed structures are configured to directly contact the eyelid of the subject.

11. The ocular treatment device of any of the preceding claims, wherein the embossed structures are configured to create pathways between the interior surface of the chamber and the eye region of the subject.

12. The ocular treatment device of any of the preceding claims, wherein the plurality of embossed structures are positioned at a distance of about 0.2 mm to about 10 mm apart from one another.

13. The ocular treatment device of any of the preceding claims, wherein the plurality of embossed structures have a height of about 0.1 mm to about 5 mm.

14. The ocular treatment device of any of the preceding claims, wherein the plurality of embossed structures are positioned in a uniform pattern on the interior surface of the chamber.

15. The ocular treatment device of any of the preceding claims, wherein each embossed structure has a shape selected from the group consisting of: a cone, a pyramid, a pentagon, a hexagon, a half sphere, a dome, a rod, an elongated ridge with round sides, and an elongated ridge with square sides.

16. The ocular treatment device of any of the preceding claims, wherein the plurality of embossed structures structure are semi-rigid.

17. The ocular treatment device of any of the preceding claims, wherein the plurality of embossed structures covers about 50 to about 100 percent of the interior surface of the chamber.

18. The ocular treatment device of any of the preceding claims, wherein the chamber is configured to provide an in vivo, tissue culture-like condition in the isolated treatment space.

19. The ocular treatment device of any of the preceding claims, wherein the isolated treatment space is characterized by a moist environment.

20. The ocular treatment device of any of the preceding claims, wherein the chamber is constructed and arranged to treat both the eye and periorbital tissue of the subject.

21. The ocular treatment device of any of the preceding claims, wherein the sealing portion comprises a mechanical or an adhesive seal.

22. The ocular treatment device of any of the preceding claims, wherein the sealing portion is configured to adhere to compromised tissue.

23. The ocular treatment device of any of the preceding claims, wherein the sealing portion is configured to adhere to burned tissue.

24. The ocular treatment device of any of the preceding claims, wherein an adhesive of the adhesive seal is substantially breathable.

25. The ocular treatment device of any of the preceding claims, wherein the adhesive is acrylic.

26. The ocular treatment device of any of the preceding claims, wherein the chamber is constructed of a substantially conformable material.

27. The ocular treatment device of any of the preceding claims, wherein the chamber is constructed of a material characterized by a predetermined oxygen permeability level.

28. The ocular treatment device of any of the preceding claims, wherein the chamber is constructed of a material that promotes oxygen transmissibility across a cornea of the subject.

29. The ocular treatment device of any of the preceding claims, wherein the chamber is constructed of polyurethane.

30. The ocular treatment device of claim 1, wherein a shape of the chamber is substantially oblong.

31. The ocular treatment device of claim 2, wherein the chamber is configured to enclose both eyes of the subject.

32. The ocular treatment device of any of the preceding claims, integrated into a facial wound chamber.

33. The ocular treatment device of any of the preceding claims, wherein the isolated treatment space is characterized by an optimized shape or volume.

34. The ocular treatment device of any of the preceding claims, wherein the chamber is constructed of a semi-transparent or substantially transparent material.

35. The ocular treatment device of any of the preceding claims, wherein the chamber is constructed of a material that is treated.

36. The ocular treatment device of any of the preceding claims, wherein the port is self- sealing.

37. The ocular treatment device of any of the preceding claims, wherein the isolated treatment space is configured to hold a predetermined volume and/or concentration of a therapeutic agent.

38. The ocular treatment device of any of the preceding claims, wherein the chamber is configured to maintain a therapeutic agent in contact with the eye and/or periorbital tissue of the subject.

39. The ocular treatment device of any of the preceding claims, wherein the isolated treatment space is configured to immerse the eye and/or periorbital tissue of the subject in a therapeutic agent.

40. The ocular treatment device of any of the preceding claims, wherein the chamber is configured to protect and/or prevent ocular injury.

41. The ocular treatment device of any of the preceding claims, configured to be removable and/or resealable.

42. The ocular treatment device of any of the preceding claims, wherein the chamber is provided in substantially sterile packaging.

43. The ocular treatment device of any of the preceding claims, wherein the chamber is disposable.

44. The ocular treatment device of any of the preceding claims, wherein the chamber is configured for single use.

45. The ocular treatment device of any of the preceding claims, wherein the chamber does not comprise a porous insert.

46. The ocular treatment device of any of the preceding claims, wherein the chamber is formed from a single sheet of material.

47. The ocular treatment device of any of the preceding claims, further comprising at least one sensor constructed and arranged to detect and/or monitor at least one of an oxygen level, temperature, and pH level within the isolated treatment space.

48. The ocular treatment device of claim 47, wherein a temperature sensor comprises a plurality of filamentary structures disposed onto a flexible substrate.

49. The ocular treatment device of claim 47, wherein an oxygen sensor comprises at least one light source and a photodetector disposed onto a flexible substrate.

50. The ocular treatment device of claim 47, wherein a pH sensor comprises at least one pH- sensitive dye incorporated into a polymer membrane and a photodetector.

51. The ocular treatment device of claim 47, wherein the at least one sensor is constructed and arranged to transmit collected data wirelessly.

52. The ocular treatment device of any of the preceding claims, further comprising at least one of a fluid trap, a pump, an exhaust port, and a suction device.

53. The ocular treatment device of any of the preceding claims, characterized by a bellows structure.

54. The ocular treatment device of any of the preceding claims, configured for clinical use in conjunction with a protective shield.

55. A method of treating bacterial keratitis in a subject, comprising:
securing the ocular treatment device of any one of claims 1-54 over a periorbital region of a subject; and
introducing a therapeutic agent to the isolated treatment space of the ocular treatment device chamber to treat the bacterial keratitis of the subject.

56. The method of any of the preceding claims, wherein the bacterial keratitis comprises Pseudomonas aeruginosa keratoconjunctivitis.

57. The method of any of the preceding claims, wherein the therapeutic agent is provided in a vehicle formulated as a liquid, suspension, gel, hydrogel, ointment, or foam.

58. The method of any of the preceding claims, wherein the therapeutic agent is formulated for sustained release.

59. The method of any of the preceding claims, wherein the therapeutic agent is an antifungal, antibiotic, anti-inflammatory, or analgesic agent.

60. The method of any of the preceding claims, wherein the antibiotic is moxifloxacin.

61. The method of any of the preceding claims, further comprising promoting a wet or moist environment in the isolated treatment space.

62. The method of any of the preceding claims, further comprising immersing a cornea of the eye of the subject in a fluid environment within the isolated treatment space.

63. The method of any of the preceding claims, wherein treatment improves corneal integrity.

64. The method of any of the preceding claims, wherein treatment reduces corneal desiccation and/or corneal scarring.

65. A method of treating an ocular condition in a subject, comprising:
securing an ocular treatment device of any one of claims 1-54 over a periorbital region of a subject; and
introducing a therapeutic agent to the isolated treatment space of the device chamber to treat the ocular condition of the subject.

66. A method of treating an ocular condition in a subject, comprising:
securing an ocular treatment device of any one of claims 1-52 over a periorbital region of a subject; and
introducing a therapeutic agent to the isolated treatment space of the device chamber to treat the ocular condition of the subject, the therapeutic agent delivered at a concentration of about 10 to about 1000 times MIC.

67. The method of any of the preceding claims, wherein the ocular condition is an ocular infection.

68. The method of claim 66 or 67, wherein the ocular infection comprises bacterial or viral keratitis.

69. The method of claim 66 or 67, wherein the ocular infection comprises Pseudomonas aeruginosa keratitis.

70. The method of any of the preceding claims, wherein the ocular condition is an ocular injury.

71. The method of any of the preceding claims, wherein the ocular injury pertains to damaged periorbital tissue.

72. The method of any of the preceding claims, wherein the ocular injury pertains to an eyelid injury.

73. The method of any of the preceding claims, wherein the ocular condition pertains to a corneal epithelial wound.

74. The method of any of the preceding claims, wherein the ocular condition pertains to a corneal ulcer.

75. The method of any of the preceding claims, wherein the ocular condition pertains to exposure keratopathy.

76. The method of any of the preceding claims, wherein the ocular condition is associated with a facial burn.

77. The method of any of the preceding claims, wherein the subject has a compromised periocular structure.

78. The method of any of the preceding claims, wherein the ocular condition pertains to a blepharoplasty procedure.

79. The method of any of the preceding claims, further comprising providing a skin graft on an eyelid of the subject to reestablish eyelid coverage.

80. The method of any of the preceding claims, wherein the graft is a partial thickness skin graft.

81. The method of any of the preceding claims, wherein the graft is a full thickness skin graft.

82. The method of any of the preceding claims, wherein the therapeutic agent is provided in a vehicle formulated as a liquid, suspension, gel, hydrogel, ointment, or foam.

83. The method of any of the preceding claims, wherein the therapeutic agent is formulated for sustained release.

84. The method of any of the preceding claims, wherein the therapeutic agent comprises an antimicrobial agent.

85. The method of any of the preceding claims, wherein the therapeutic agent is an antifungal, antibiotic, anti-inflammatory, or analgesic agent.

86. The method of any of the preceding claims, wherein the antibiotic is vancomycin.

87. The method of any of the preceding claims, wherein the antibiotic is moxifloxacin.

88. The method of any one of claims 55-65, wherein the therapeutic agent is delivered at a concentration of about 10 to about 1000 times MIC.

89. The method of any of the preceding claims, the therapeutic agent is delivered at a concentration of up to 100 times MIC.

90. The method of any of the preceding claims, the therapeutic agent is delivered at a concentration of at least 100 times MIC.

91. The method of any of the preceding claims, further comprising promoting a wet environment in the isolated treatment space.

92. The method of any of the preceding claims, further comprising immersing a cornea of the eye of the subject in a fluid environment within the isolated treatment space.

93. The method of any of the preceding claims, further comprising introducing oxygen to the device chamber.

94. The method of any of the preceding claims, further comprising introducing a saline solution or a media solution to the device chamber.

95. The method of any of the preceding claims, further comprising introducing a growth factor and/or cells, to the ocular treatment device chamber.

96. The method of any of the preceding claims, wherein treatment improves corneal integrity.

97. The method of any of the preceding claims, wherein treatment reduces corneal desiccation and/or corneal scarring.

98. The method of any of the preceding claims, wherein treatment slows or prevents progression of ocular wound depth.

99. The method of any of the preceding claims, wherein surrounding tissue and/or tissue under an adhesive rim of the ocular wound chamber is unharmed.

100. The method of any of the preceding claims, wherein treatment does not result in a change to corneal epithelium, stroma, or endothelium integrity.

101. The method of any of the preceding claims, wherein treatment does not result in a change to corneal thickness, intraocular pressure, or corneal surface morphology, i.e., no abrasion.

102. The method of any of the preceding claims, wherein long-term vision damage is prevented.

103. The method of any of the preceding claims, wherein eyelid retraction and/or eyelid scarring is reduced.

104. The method of any of the preceding claims, wherein corneal swelling, infiltrate, or neovascularization is avoided.

105. The method of any of the preceding claims, wherein ocular infection is resolved within fourteen days.

106. The method of any of the preceding claims, wherein the device chamber is left in place for up to 7 days prior to replacement.

107. The method of any of the preceding claims, wherein therapeutic agent within the isolated treatment space is replaced at a minimum frequency of about every 24 hours.

108. The method of any of the preceding claims, further comprising identifying the subject as having an ocular condition.

109. The method of any of the preceding claims, further comprising decontaminating and/or debriding the ocular wound.

110. The method of any of the preceding claims, further comprising imaging the eye of the subject.

111. The method of any of the preceding claims, further comprising monitoring the environment of the eye.

112. The method of claim 111, wherein monitoring the environment of the eye comprises measuring at least one of a temperature, oxygen, and pH level within the isolated treatment space.

113. The method of any of the preceding claims, wherein treatment with the ocular treatment device increases a blood plasma concentration of the therapeutic agent in the subject relative to administration of the therapeutic agent alone.

114. The method of any of the preceding claims, further comprising applying negative pressure therapy to the eye region.

115. The method of any of the preceding claims, wherein the device chamber is secured to the subject after onset of an ocular infection or an ocular injury.

116. The method of any of the preceding claims, wherein the device chamber is applied at a point of injury for stabilization.

117. The method of any of the preceding claims, wherein the device chamber is secured prophylactically.

118. The method of any of the preceding claims, wherein the ocular condition of the subject is treated in conjunction with a facial wound of the subject.
